(19) United States
(12) Patent Application Publication
(10) Pub. No.: US 2006/0094865 A1
(43) Pub. Date: May 4, 2006

(54) Intraoperative method for isolating and concentrating autologous growth factors and for forming residual autologous growth factor compositions

(76) Inventors: Terri A. Kapur, Stoughton, MA (US); Stephen Jaquith, Bellingham, MA (US); Shannon S. Webster, Quincy, MA (US); Sudhakar Kadiyala, Newton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/977,858

(22) Filed: Oct. 29, 2004

(51) Int. Cl. C07K 14/475; B04B 11/04

(52) U.S. Cl. 530/399; 435/287.1; 494/6

(57) ABSTRACT

This invention is concerned with methods and devices for isolation or concentration of autologous growth factors; particularly autologous growth factors derived from blood or bone marrow or other bodily fluid/cell compositions in an intraoperative manner. Additionally, this invention provides specifically tailored growth factor residual compositions resulting from removal of one or more growth factors from a composition.

### BACKGROUND OF THE INVENTION

1. Field of the Invention

This invention is concerned with isolation or concentration of autologous growth factors, particularly autologous growth factors derived from blood or bone marrow in an intraoperative manner. Additionally, the invention relates to modified residual plasma or marrow compositions which may have one or more components removed from the blood or bone marrow aspirate. This invention in all its aspects is applicable to other bodily fluid compositions particularly those containing cells.

2. Related Art

Numerous platelet rich plasma (PRP) preparations derived from blood exist both in commercial practice and under development. In these various methods, different techniques including filtration or centrifugation are employed to concentrate the platelets. In this process, other blood components such as excess plasma and red blood cells are removed. In certain methods, other components, such as white blood cells, may also be concentrated with the platelets, either intentionally or unintentionally.

PRP contains mixtures of various growth factors and other protein and non-protein components. A non-exhaustive list of such growth factors enzymes, cytokines and chemokines such as collagenase, interleukins, tumor necrosis factor (TNF), transforming growth factor (TGF), insulin like growth factor (IGF), C5a (compliment), serotonin, von Willebrand Factor, epidermal growth factor (EGF), fibronectin, fibrinogen, histamine, platelet derived growth factor (PDGF), vascular endothelial growth factor (VEGF), adiponectin, transferrin, and lactoferrin.

Bone Marrow aspirate may contain many of the same or a similar list of proteins as in PRP in addition to stem cells. Additional proteins include but not limited to those associated with mesenchymal stem cells such as: Bone Morphogenetic Proteins (BMP), leukaemia inhibitory factor (LIF), ciliary neurotrophic factor (CNTF), mRNAs.

Some of these components may be undesirable for specific indications and may reduce the effectiveness of PRP or marrow in these indications/sites. Alternately, it may be desirable to remove specific growth factors from the PRP or marrow.

Currently, there is no known product available to isolate specific individual autologous growth factors. Additionally, purified growth factors can only be obtained in a laboratory (non-intraoperatively). The invention disclosed herein will allow intraoperative isolation, and therefore concentration, of specified autologous growth factors with the ability to further enhance the desired function of the residual blood plasma marrow, or other bodily fluid compositions by selective removal of components from the material to make them more effective.

### BRIEF DESCRIPTION OF THE DRAWINGS

FIGS. 1a-1d depict one embodiment of this invention related to forming residual autologous growth factor compositions; and

FIGS. 2a-2b depict another embodiment of this invention related to isolating autologous growth factors.

### SUMMARY OF THE INVENTION

One embodiment of this invention relates to a method for isolating or concentrating autologous growth factors comprising the steps of:
a) providing a composition containing autologous growth factors;
b) centrifuging the composition to form a fraction rich in autologous growth factors;
c) centrifuging to contact the fraction rich in autologous growth factors with a substrate containing an affinity coating or affinity material specific to remove a select growth factor or component from the fraction and to form a residual fraction of autologous growth factors; and
d) recovering the residual fraction of autologous growth factors.

Another embodiment of this invention relates to a method for isolating or concentrating autologous growth factors comprising the steps of:
a) providing a composition rich in autologous growth factors;
b) centrifuging to contact the fraction rich in autologous growth factors with a substrate containing an affinity coating or affinity material specific to remove a select growth factor or component from the fraction and to form a residual fraction of autologous growth factors; and
c) recovering the residual fraction of autologous growth factors.

Yet other embodiments of this invention relate to the above method further comprising the steps of:
e) centrifuging to contact the affinity coating or affinity material with an elution buffer to release the growth factor or component bound by the affinity coating or affinity material; and
f) recovering the elution buffer containing the released growth factor or component.

The invention also relates to devices comprising a centrifuge and multi-chambered, dual-chambered, or single-chambered containers comprising an affinity column for selectively removing one or more components to isolate or concentrate autologous growth factor compositions. Additionally, the aforementioned chambered containers as containers in and of themselves form other embodiments of the present invention.

An advantage of the present invention is that not only is there provided an intraoperative method and device for isolating or concentrating autologous growth factors but also that the method can be performed in a manner in which selected items may be removed or tailored to enhance the desired function of the residual compositions such as those derived from blood or bone marrow by selective removal of components from the material to make them more effective and better suited for performance for a desired application.

### DETAILED DESCRIPTION OF PREFERRED EMBODIMENTS OF THE INVENTION

One embodiment of the present invention describes a method and device by which individual growth factors can be selectively removed from platelets utilizing a sample of autologous blood. This is beneficial to allow the use of one or more specific growth factors for therapeutic purposes. For instance, if one growth factor contained in platelets is understood to hinder bone formation, it can be removed via this technique. Upon removal, the mixture of the remaining growth factors (residual) could be implanted to enhance healing. Alternatively, if one or more specific growth factors has been identified to further a healing process, they can be isolated, and subsequently implanted.

When working with whole blood for example, this process involves a sample of patient blood, a multiple-chambered container which could be (disposable), and a centrifuge. It can be performed intraoperatively in approximately 20 minutes. Any further description of the technique is meant only to aid in scientific explanation of the process, not to restrict the design of the apparatus.

In general, the steps of the process are as follows as it relates to processing whole blood:

1. A sample of patient blood is drawn.

2. The blood is placed into a first chamber of the container.

3. Centrifugation separates the blood components and the platelets/plasma are decanted into a second chamber of the container (which may already contain a volume of degranulation agent or growth factor releasant agent).

4. A quick, hard spin (centrifugation) pulls platelets to the bottom of the second chamber and a slower cycle (centrifugation) will decant the plasma (containing the growth factors) into a third chamber of the container. The third chamber contains an affinity column specific for removal or isolation of one or more growth factors or other component(s).

5. The growth factor(s) or other component(s) of choice are retained over the affinity column while the remainder of the plasma is eluted to the bottom of the third chamber of the container.

6. Further centrifugation decants the plasma effluent into a fourth chamber of the container through a side pathway (not to interfere with the affinity column) to provide a residual composition of growth factors.

7. If recovery of the isolated growth factor(s) or other component(s) on the affinity columns is desired, a further centrifugation of sufficient force to release an elution buffer contained in a previously sealed reservoir in the third chamber is performed. The released buffer upon contact with the affinity column will elute the growth factors from the affinity column to form a composition containing the growth factor(s) or other component(s).

8. A syringe equipped with a desalting cartridge will allow removal of the isolated growth factor elution mixture.

9. The isolated mixture (or the eluted plasma if desired) can be used as an autologous therapeutic agent.

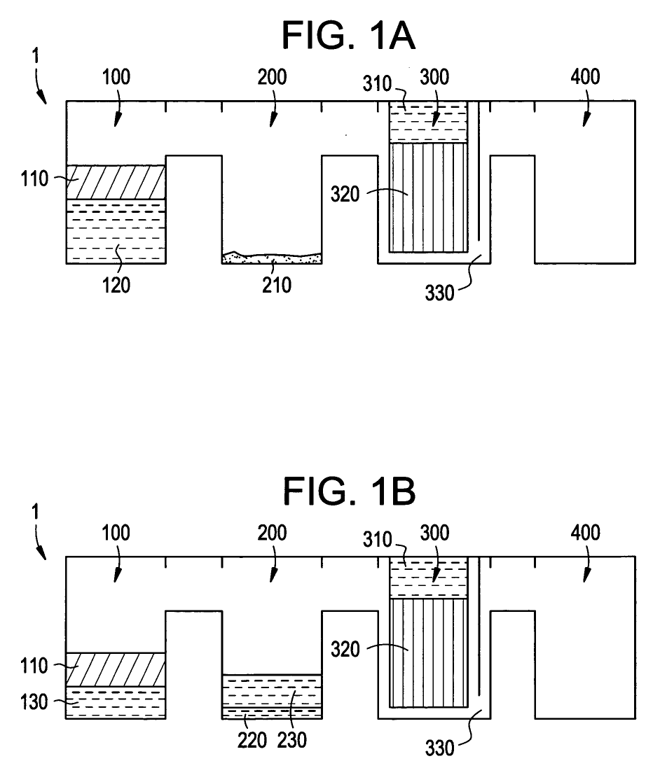

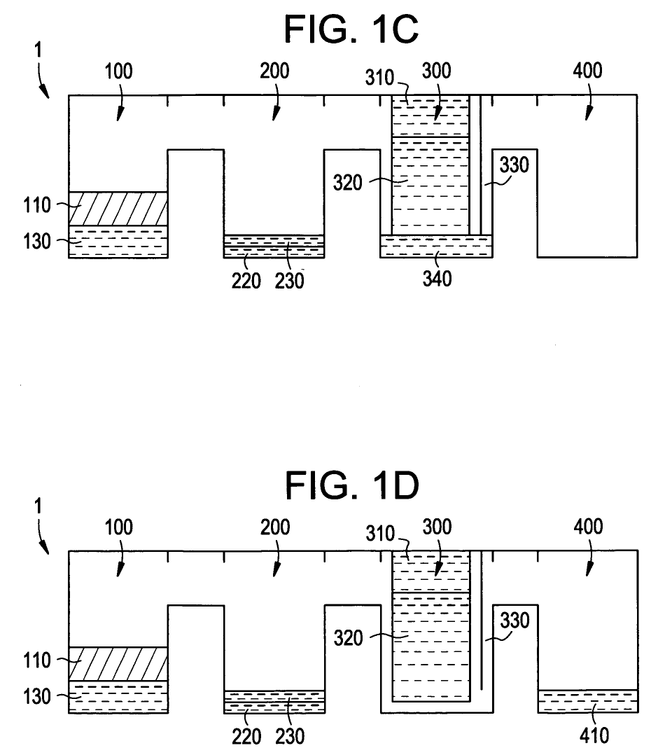

One embodiment of the device of this invention is shown in FIG. 1a. Device 1 comprises chambers 100, 200, 300, and 400 which are interconnected. Chamber 100 serves as a container for acceptance of a material 120 that contains autologous growth factors. Non-limitative examples of such materials include blood, bone marrow aspirate, tumor aspirate, spinal fluid, lymphatic fluid, other interstitial fluid and essentially any other bodily fluid/cell mixtures present in the body. Chamber 100 is also depicted to contain an optional floating shelf 110. Floating shelf 110 may be used to help with the initial centrifugation of material 120 into its component parts and floating shelf 110 is designed to have a specific gravity between the components that material 120 will be separated into after centrifugation. It is envisioned that chamber 100 may contain multiple floating shelves depending on the number of components layers that the centrifugation may form. Examples of floating shelves (separator disks) may be found in WO 01/83968 A1 of Harvest Technologies Corporation, the disclosure of which is incorporated by reference.

Chamber 200 of device 1 is a receiver of the decanted fluid coming from chamber 100. Chamber 200 may contain a degranulation agent 210 (growth factor releasant) in order to release or enhance release of growth factors from the decanted fluid of chamber 100.

Suitable examples of growth factor releasant 210 include but are not limited to positively charged compounds, many mast cell secretions (in general), more specifically: thrombin, Immunoglobulin Gs, non-ionic monomeric iodinated X-ray agents, neuropeptides, calcium ions, anaphylotoxins (compliment), platelet activation factor (PAF), codeine, light, and alcohol.

While the releasants many have varying degrees of potency, one skilled in the art would appreciate that such releasants should work on bone marrow aspirate as well as blood and other bodily fluid/cell mixtures present in the body as described above, for example.

Chamber 300 contains optional reservoir 310, affinity column 320 and plasma elution channel 330. Affinity column 320 is a separation device which is designed to separate desired growth factor(s) or component(s) from the fluid entering chamber 300 from chamber 200. For example, affinity column 320 may contain a substrate with a coating or a material specifically designed to bind the desired growth factor or component. Those skilled in the art shall know that examples of suitable coatings that may contain the antibodies or peptides for retaining the desired growth factor or component. Plasma elution channel 330 serves as a conduit for fluid to be transferred from chamber 300 to chamber 400.

It should be noted that there are many proteins that are present at different levels in bodily fluid/cell mixtures that can also be separated and may not yet be discovered. As will be apparent to one skilled in the art, the present invention is flexible enough to address virtually any protein present in these bodily fluid/cell mixtures. It is limited only by the availability of a capture mechanism for the affinity column. The capture mechanisms can be specific such as peptides, antibodies, proteins, and receptor-protein interactions, or non-specific such as charge-charge or hydrogen bonding interactions.

Reservoir 310, when present, contains an elution buffer that is released to remove the growth factor or component attached to affinity column 320. In a preferred embodiment, the elution buffer is sealed with a film that is sensitive to gravitational forces and which will release the elution buffer at a predetermined centrifugation speed. Alternately, if reservoir 310 is not present, elution buffer may be added manually to the top of chamber 300 through a port (not shown).

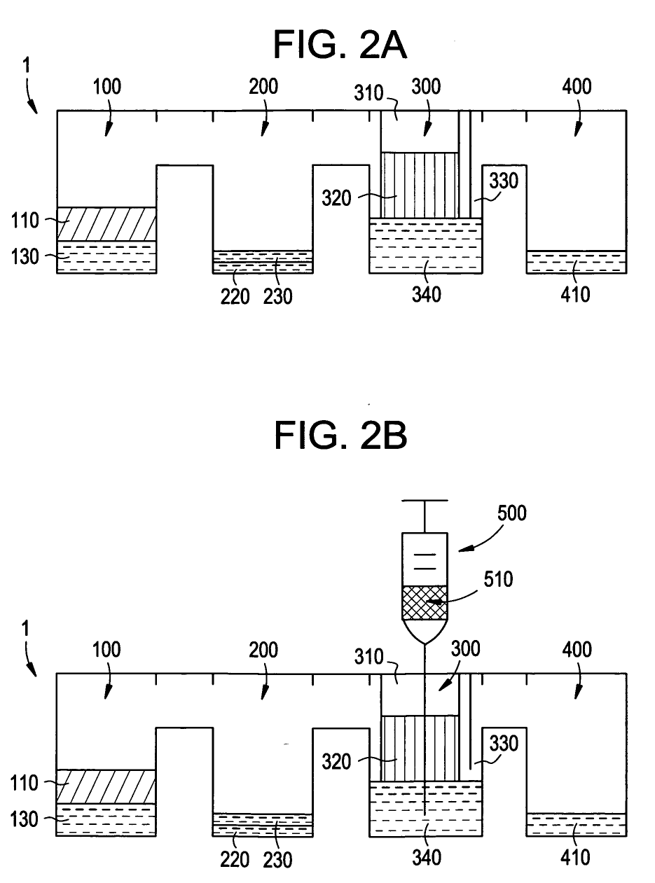

Chamber 400 is a holding container for the (modified) residual autologous growth factor composition 410 (shown in FIGS. 1d, 2a and 2b).

In operation (still referring to FIG. 1a) to obtain the residual autologous growth factor composition 410 (not shown) and for the embodiment where whole blood is being processed, whole blood 120 is placed in chamber 100 of device 1. If degranulation agent or releasant agent 210 is to be used, it is placed in chamber 200. With device 1 in a centrifuge (not shown), centrifugation is conducted to separate whole blood 120 into its components of red blood cells and platelet rich plasma in chamber 100 and after slowing the centrifugation down to decant the platelet rich plasma into chamber 200. Referring to FIG. 1b, (i.e., the end point of the first centrifugation and after decanting) shows red blood cells 130 remaining in chamber 100 and a fraction of red blood cells 220 and degranulated platelet plasma 230 in chamber 200.

FIG. 1c represents the endpoint in the process after further centrifugation and decantation of the degranulated platelet plasma 230. In this process step, degranulated platelet plasma 230 contacts affinity column 320 in which a desired growth factor or other component is removed leaving a modified platelet rich plasma residual 340.

FIG. 1d represents the transfer of modified degranulated platelet plasma 340 from chamber 300 after centrifugation into chamber 400 where is labeled as residual degranulated platelet plasma 410. Residual plasma 410 now tailored to a desired composition, may be removed from device 1 for use in its intended application.

Recovery of specific autologous growth factors or other components may further be completed at this point. Referring to FIG. 2a, the resulting step depicted in FIG. 1 d is further centrifuged at a speed sufficient to break the film encapsulating the elution buffer contained in reservoir 310. The released elution buffer contacts affinity column 320 resulting in release of the growth factor(s) contained or other component(s) from affinity column 320 into the elution buffer thereby forming composition 340 of growth factor(s) or other component(s) and elution buffer.

FIG. 2b depicts recovery of composition 340 by introduction of syringe 500. The needle of syringe 500 is inserted into chamber 300 and into composition 340. As composition 340 is withdrawn from chamber 300, it passes over optional desalting cartridge 510 in syringe 500 to removes salts that are typically part of the elution buffer. Once this point is reached, the contents syringe 500 may be injected where desired. In certain instances desalting cartridge 510 may need to be removed prior to injection. Alternately, contents of syringe 500 may be transferred to a sterile field, such as by transferring the contents of syringe 500 into a sterile cup on a sterile field and then surgeon could apply the mixture using a spray applicator or a graft delivery system.

It will be understood by those skilled in the art that the device and method of this invention may be an embodiment which is a device of less than 4 chambers, particularly if PRP is used as the starting component instead of whole blood and therefore there would be no need for a first separation chamber to separate the whole blood into its PRP component. In particular, if particular growth factors are only required to be removed from PRP or bone marrow aspirate, only an affinity column may be required and as such a single-chambered container is contemplated by this invention engageable with a centrifuge, comprising an affinity column for selectively removing one or more components of a composition comprising autologous growth factors. In another embodiment where only a particular growth factor may be desired to be deactivated, a deactivating agent may be included in the composition specific to the targeted growth factor. Some illustrative examples of these concepts are found in the following, non-limitative examples of methods and devices.

Another aspect of this invention relates to the understanding that removal of components, such as specific growth factors from PRP, bone marrow aspirate, or other bodily fluid/cell mixtures may allow the resulting residual compositions to function more effectively in specific indications. The rationale behind the idea is that since the specific concentration and ratios of growth factors and other components in platelets and serum have evolved to function in a wide variety of injury sites, and thus are not optimized for any specific site or indication. Thus removal (or substantial reduction) of a specific component from the mixture found, for example, in PRP or bone marrow aspirate may enhance the functional activity of the PRP or aspirate for that indication/site. While certain components, such as red blood cells, are removed during the processing for PRP, there is no specific attempt to remove components that are an integral part of the preparation to enhance it for specific indications/sites.

The specific components that are to be reduced substantially in concentration may be specific growth factors, such as transforming growth factor-β (TGF-β) or other components such as fibronectin. The component to be reduced may arise specifically from the platelets or non-platelet sources. The preferred preparation in these instance is autologous PRP.

The above strategy may also be applied to other physiologic preparations such as bone marrow aspirates and other bodily fluid/cell compositions.

### EXAMPLES

### Example 1

PRP is made from 55 cc of whole blood using the Symphony® system available from DePuy Spine, Raynham, Mass., USA. The prepared PRP is run through a column that contains antibodies to epidermal growth factor (EGF). Majority of the EGF binds to the antibodies and is removed from the PRP.

### Example 2

PRP is made from 55 cc of whole blood using the Symphony® system. A modified version of TGF-β binding protein that irreversibly binds to TGF-β is added to the PRP. Thus, the majority of the TGF-β is irreversibly bound and is not available for physiologic action.

### Example 3

PRP is made from 55 cc of whole blood using the Symphony® system. The container in which the PRP is collected is coated with peptides that specifically bind to fibronectin fragments. Thus, the fibronectin fragments is substantially removed from the preparation. Such a preparation may be beneficial for cartilage or intervertebral disc applications where fibronectin fragments may have undesirable consequences.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A method for isolating or concentrating autologous growth factors comprising the steps of:
a) providing a composition containing autologous growth factors;
b) centrifuging the composition to form a fraction rich in autologous growth factors;
c) centrifuging to contact the fraction rich in autologous growth factors with a substrate containing an affinity coating or affinity material specific to remove a select growth factor or component from the fraction and to form a residual fraction of autologous growth factors; and
d) recovering the residual fraction of autologous growth factors.

2. The method of claim 1, wherein the composition containing autologous growth factors is whole blood.

3. The method of claim 1, wherein the composition containing the autologous growth factors is platelet rich plasma.

4. The method of claim 1, wherein the composition containing the autologous growth factors is bone marrow aspirate.

5. A method for isolating or concentrating autologous growth factors comprising the steps of:
a) providing a composition rich in autologous growth factors;
b) centrifuging to contact the fraction rich in autologous growth factors with a substrate containing an affinity coating or affinity material specific to remove a select growth factor or component from the fraction and to form a residual fraction of autologous growth factors; and
c) recovering the residual fraction of autologous growth factors.

6. The method of claim 5, wherein the composition rich in autologous growth factors is platelet rich plasma.

7. The method of claim 5, wherein the composition rich in autologous growth factors is bone marrow aspirate or is derived from bone marrow aspirate.

8. The method of claim 5, wherein the composition rich in autologous growth factors is derived from spinal fluid.

9. The method of claim 1 or 5, further comprising the steps of:
e) centrifuging to contact the affinity coating or affinity material with an elution buffer to release the growth factor or component bond by the affinity coating or affinity material; and
f) recovering the elution buffer containing the released growth factor or component.

10. A device for isolating or concentrating autologous growth factors comprising:
a) a centrifuge; and
b) a multi-chambered container for use and engagement with the centrifuge, wherein the multi-chambered container comprises:
i) a first chamber for receipt of a composition containing autologous growth factors;
ii) a second chamber for receipt of a fraction rich in autologous growth factors from the composition of the first chamber after centrifugation;
iii) a third chamber comprising an affinity column for selectively removing one or more components from the fraction decanted from the second chamber and for forming a residual fraction; and
iv) a fourth chamber for receipt of the residual fraction from the third chamber after centrifugation.

11. The device of claim 10, wherein the affinity column comprises antibodies or peptides to bind epidermal growth factor.

12. The device of claim 10, wherein the affinity column comprises antibodies or peptides to bind TGF-β.

13. The device claim 10, wherein the affinity column comprises antibodies or peptides to bind fibronectin.

14. The device of claim 10, wherein the affinity column comprises antibodies or peptides to bind lactoferrin.

15. A multi-chambered container engagable with a centrifuge comprising:
i) a first chamber for receipt of a composition containing autologous growth factors;
ii) a second chamber for receipt of a fraction rich in autologous growth factors from the composition of the first chamber after centrifugation;
iii) a third chamber comprising an affinity column for selectively removing one or more components from the fraction decanted from the second chamber and for forming a residual fraction; and
iv) a fourth chamber for receipt of the residual fraction from the third chamber after centrifugation.

16. A device for isolating or concentrating autologous growth factors comprising:
a) a centrifuge; and
b) a multi-chambered container for use and engagement with the centrifuge, wherein the multi-chambered container comprises:
i) a first chamber for receipt of a composition rich in autologous growth factors and comprising an affinity column for selectively removing one or more components of the composition and for forming a residual fraction; and
ii) a second chamber for receipt of the residual fraction from the first chamber after centrifugation.

17. A multi-chambered container engagable with a centrifuge comprising:
i) a first chamber for receipt of a composition rich in autologous growth factors and comprising an affinity column for selectively removing one or more components of the composition and for forming a residual fraction; and
ii) a second chamber for receipt of the residual fraction from the first chamber after centrifugation.

18. A device for isolating or concentrating autologous group factors comprising:
a) a centrifuge; and
b) a single-chambered container for use and engagement with the centrifuge, wherein the container comprises an affinity column for selectively removing one or more components of a composition comprising autologous growth factors.

19. A single-chambered container engagable with a centrifuge wherein the container comprises an affinity column for selectively removing one or more components of a composition comprising autologous growth factors.
